(19) United States
(12) Patent Application Publication
(10) Pub. No.: US 2005/0182124 A1
(43) Pub. Date: Aug. 18, 2005

(54) Ester bacteriostat for stabilizing cosmetics

(75) Inventor: Giovanni Razzano, Fino (IT)

Correspondence Address: THE FIRM OF KARL F ROSS, 5676 RIVERDALE AVENUE, PO BOX 900, RIVERDALE (BRONX), NY, 10471-0900, US

(73) Assignee: VAMA FARMACOSMETICA S.r.l.

(21) Appl. No.: 10/941,629

(22) Filed: Sep. 15, 2004

(30) Foreign Application Priority Data
Feb. 13, 2004 (IT) MI2004A000244

(51) Int. Cl. A61K 31/4015

(52) U.S. Cl. 514/423

(57) ABSTRACT

A bacteriostat consisting of phenoxyethanol ester of 2-pyrrolidon-5-carboxylic acid (PCA) of formula 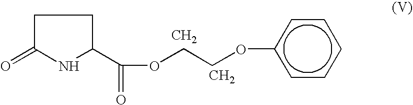 With respect to conventional bacteriostats, the one according to the invention offers the advantage of preventing microbiological pollution, without causing reactive responses from the skin. The bacteriostat of the invention also offers higher bacteriostatic activity.

### FIELD OF THE INVENTION

The present invention relates to a new bacteriostat for preparations for topical use, in particular for dermopharmaceutical and cosmetic use. The invention also extends to the preparation that contains this bacteriostat and to the use thereof in the dermopharmaceutical and cosmetic field. The field of the invention is that of bacteriostats used to prevent microbiological pollution of cosmetic and dermopharmaceutical preparations (for example emulsions and creams), so as to increase the stability and useful time of these preparations, also ensuring the safety thereof during use. Conventional bacteriostats, suitable for conserving the preparations that contain them, have more or less serious toxicological interference with skin physiology.

The main purpose of the present invention is that of providing a new bacteriostat capable of offering, in addition to a more effective bacteriostatic activity, also excellent skin tolerability.

These and other purposes are accomplished with the bacteriostat and the preparations according to claims 1, 7 and 8, respectively. Preferred embodiments of the invention are given by the remaining claims.

With respect to conventional bacteriostats, the one according to the invention offers the advantage of preventing microbiological pollution, without causing reactive responses from the skin.

A further advantage of the bacteriostat of the invention is represented by the higher bacteriostatic activity offered by it. The last but not least advantage of the bacteriostat of the invention is that it also carries out bactericide action.

These and other advantages, purposes and characteristics are clear from the following description of some preferred embodiments of the invention, described hereafter purely as a nonlimiting example.

The bacteriostat of the invention consists of the product of the esterification reaction of a carboxylic acid of formula
R—COOH (I)
Where R can be a saturated or unsaturated aliphatic chain, or else a saturated or unsaturated cyclic or heterocyclic structure, with an alcohol with bacteriostatic action of formula 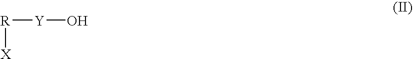
if R is a saturated or unsaturated aliphatic chain, Y is H and X is Br or I;
if R is a saturated or unsaturated cyclic or heterocyclic structure, Y is —OCH₂CH₂— and X is H.

The preferred carboxylic acid for the invention is 2-pyrrolidon-5-carboxylic acid (PCA) of formula

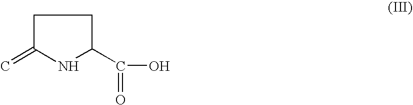

Other carboxylic acids that can be used for the purposes of the invention are glycolic acid, salicylic acid, sorbic acid, azelaic acid, tartaric acid, citric acid and coconut fatty acids C₈₋₁₀.

Also preferred for the invention. is 2-phenoxyethanol alcohol of formula

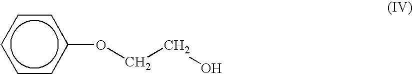

Other alcohols that can be used for the purposes of the invention are alcohols with bacteriostatic effect 2-Br-ethanol, 2-I-ethanol and tetrahydrofurfuryl alcohol.

Alternatively epichlorohydrin or epibromohydrin may be reacted with carboxylic acids to produce these esters.

The preferred bacteriostat for the invention consists of the phenoxyethanol ester of PCA Laving the formula 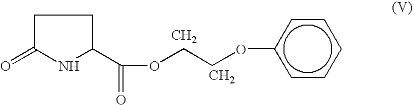
Obtained from the esterification reaction of phenoxyethanol alcohol of formula (IV) with the PCA of formula (III).

The bacteriostat of the invention also advantageously carries out a bactericide activity.

The skin compatibility of the bacteriostat according to the invention, in terms of anergic response and of primary Irritation. was tested with the following patch tests.

### Patch Test

A panel of 25 healthy volunteers was subjected to the application onto the back of about 20 μg of solid product with the use of a “finn chamber” (patch test). After 48 hours the “finn chamber” was removed and the skin reactions after 15 mins, 1 hr and 24 hrs from removal were clinically evaluated.

### EXAMPLE 1

### Comparison

Tn the example, shown here for comparative purposes, phenoxyethanol alcohol of formula (IV) was used. The hacteriostat proved to be moderately irritating.

### EXAMPLE 2

### Invention

To carry out the test protocol described above a bacteriostat consisting of phenoxyethanol ester of PCA of formula (V) of the invention was used. The bacteriostat proved not to be irritating and did not sensitize the skin.

The bacteriostat of the invention is advantageously used to stabilize cosmetic preparations, for example emulsions like creams, fluid emulsions, lotions and the like, as well as to stabilize dermopharmaceutical preparations like creams, lotions and gels.

With the purpose of evaluating the bacteriostatic activity of the bacteriostats of the invention, the following MIC (Minimal Inhibition Concentration) tests were carried out on bacterial colonies of “Pseudomonas aeruginosa” (Pa) and of “Staphylococcus aureus” (Sa), respectively with pure phenoxyethanol of formula (IV) and with phenoxyethanol ester of PCA of formula (V) of the invention.

The results of the tests are collected in the following Table:

TABLE

| | phenoxyethanol | PCA Ester |
|---|---|---|
| BACTERIA | (ppm) | (ppm) |
| Pa | 3200 | 1000 |
| Sa | 6400 | 1000 |

As can be seen from this table, the ester of formula (V) of the invention offers the same bacteriostatic activity as phenoxyethanol, but with the use of lower concentrations of the active ingredient.

1. Bacteriostat for preparations for topical use, characterized in that it consists of an ester.

2. Bacteriostat according to claim 1, characterized in that said ester consists of the product of the reaction of a carboxylic acid with an alcohol of formula 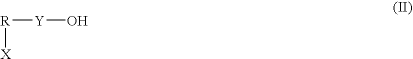
Wherein:
if R is a saturated or unsaturated aliphatic chain, Y is H and X is Br or I;
if R is a saturated or unsaturated cyclic or heterocyclic structure, Y is —OCH₂CH₂— and X is H.

3. Bacteriostat according to claim 2, characterized in that said eater is the product of the reaction of said alcohol of formula, (II) with 2-pyrrolidon-5-carboxylic acid (PCA) of formula:

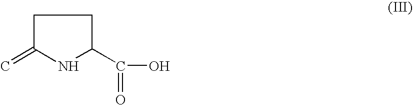

4. Bacteriostat according to claim 3, characterized in that said alcohol is phenoxyethanol alcohol of formula 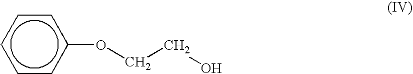
and in that said bacteriostat consists of phenoxyethanol ester of PCA of formula:

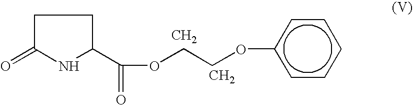

5. Bacteriostat according to claim 2, characterized in that said carboxylic acid consists of glycolic acid, salicylic acid, sorbic acid, azelaic acid, tartaric acid, citric acid and coconut fatty acids C₈₋₁₀.

6. Bacteriostat according to claim 2, characterized in that said alcohol consists of 2-Br-ethanol, 2-I-ethanol or tetrahydrofurfuryl alcohol.

7. Bacteriostat according to claim 1, characterized in that it also carries out a bactericide action.

8. Dermopharmaceutical preparation, characterized in that it comprises at least one bacteriostat according to claim 1.

9. Cosmetic preparation, characterized in that it comprises at least one bacteriostat according to claim 1.

10. Use of the bacteriostat according to claim 1 for making dermopharmaceutical and cosmetic preparations.
